(19) United States
(12) Patent Application Publication
(10) Pub. No.: US 2023/0082811 A1
(43) Pub. Date: Mar. 16, 2023

(54) ADAPTATION OF PLATFORM HOSTS TO IGF- MEDIA

(71) Applicant: Amgen Inc., Thousand Oaks, CA (US)

(72) Inventors: Kristine Marie DARIS, Newbury Park, CA (US); Huong Thi Ngoc LE, Newbury Park, CA (US); Eric GISLASON, Newbury Park, CA (US); Trent Phillip MUNRO, Kenmore QLD (AU)

(21) Appl. No.: 17/930,810

(22) Filed: Sep. 9, 2022

Related U.S. Application Data
(60) Provisional application No. 63/242,623, filed on Sep. 10, 2021.

(51) Int. Cl. C12P 21/02; C12N 5/00; C12N 15/90; C07K 16/00

(52) U.S. Cl. CPC C12P 21/02; C12N 5/00; C12N 15/907; C07K 16/00; C12N 2501/105

(57) ABSTRACT

Methods producing a recombinant protein of interest in a mammalian cell culture in media lacking IGF-1 are provided. Methods for producing mammalian cells capable of growing in media lacking IGF-1 are also provided.

### FIELD OF THE INVENTION

The present invention relates generally to methods for adapting mammalian cell lines to cell culture media having reduced amounts of Insulin-like Growth Factor (IGF-1) and use of these cells to produce recombinant proteins.

### BACKGROUND OF THE INVENTION

Due to their broad applications, biologics are used worldwide in a variety of applications, such as therapeutics and diagnostics Mammalian cell lines are the predominant expression systems for these biologics, with Chinese hamster ovary (CHO) cells being the predominate cellular factory. See Lalonde et al., 2017, J Biotechnol 251:128-140. Particularly with the advent of biosimilars, speed-to-market and cost-efficiency are now more important than ever before.

The costs of manufacturing biologics are high due to their complexity of production utilizing a multistep process involving the selection of optimal cell lines, culturing production cells in large quantities, and purification of the desired biologic from the cell harvest. While these costs are decreasing due to improvements in all facets of production, their costs can still be prohibitive in their widespread adoption as front-line therapies.

In order to make biological therapeutics more accessible to patients, decreasing the cost of goods for the manufacturing process is an attractive proposition. One area of significant cost is the cell culture medium used in the drug substance process. IGF-1 is a critical protein supplement that supports cell growth through signaling of the Insulin-like Growth Factor/Insulin Receptor (IGFR/IR) pathway; however, it makes up a significant proportion of the raw material costs for the medium.

As such, there is a need to reduce costs associated with recombinant protein production from host cells. One way to achieve this objective is to reduce the cost of goods by reducing or eliminating the need for certain cell culture media supplements such as IGF-1. Enhanced Insulin-like Growth Factor-1 Receptor (IGF-1R) expression has been seen in mesenchymal stem cells through the supplementation of cell culture media with platelet-derived growth factor BB. See U.S. Patent Application Publication No. US20200245388. Constitutive expression of IGF-1R has also been employed using expression vectors. See U.S. Provisional Patent Application No. 63/108,084. Gradual adaption of host cell lines has been used to adapt cells to protein-free and lipid-free media. See U.S. Pat. No. 9,340,814.

There still exists a need for host cell lines with reduced or no requirements for IGF-1 supplementation that produce recombinant proteins with minimal impact on growth and productivity. Such cell lines would benefit the process development of biologics.

### SUMMARY OF THE INVENTION

The present disclosure provides a method for producing a protein of interest from a mammalian cell culture comprising (a) culturing a mammalian cell in a second cell culture media having 0.05 mg/L or less Insulin Like Growth Factor (IGF-1) to express the protein of interest, wherein the mammalian cell has been directly adapted to grow in a first cell culture media having 0.03 mg/L or less IGF-1 and comprises a heterologous nucleic acid encoding the protein of interest; and (b) recovering the protein of interest produced by the mammalian cell.

In certain embodiments, the second cell culture media contains 0.03 mg/L or less IGF-1. In certain embodiments, the first cell culture media contains no IGF-1. In certain embodiments, the second cell culture media contains no IGF-1.

In certain embodiments, the mammalian cell which has been directly adapted has a growth rate comparable to a mammalian cell of the same lineage that has not been directly adapted. For example, a directly adapted mammalian cell can have a doubling time less than 30 hours, such as between 20 to 30 hours.

In certain embodiments, employing the methods described herein, the titer of the expressed protein of interest is at least 50 mg/L at day 10 of the culture.

In certain embodiments, the protein of interest is an antigen binding protein. In certain embodiments, the protein of interest is selected from the group consisting of monoclonal antibodies, bi-specific T cell engagers, immunoglobulins, Fc fusion proteins and peptibodies.

In certain embodiments, the mammalian cell culture process utilizes a fed-batch culture process, a perfusion culture process, or combinations thereof.

In certain embodiments, the mammalian cell culture is established by inoculating a bioreactor of at least 100 L with at least 0.5×10⁶ to 3.0×10⁶ cells/mL in a serum-free culture media with 0.03 mg/L or less IGF-1. In certain aspects of this embodiment, the bioreactor is at least 500 L or at least 2000 L.

In certain embodiments, the mammalian cells are Chinese Hamster Ovary (CHO) cells. In certain embodiments, the CHO cells are deficient in dihydrofolate reductase (DHFR) or are a glutamine synthetase knock out (GSKO).

In certain embodiments, the recovered protein of interest is purified and formulated in a pharmaceutically acceptable formulation.

The present disclosure also provides purified, formulated protein of interest prepared using the methods described herein.

The present disclosure also provides a method for directly adapting a mammalian cell to IGF− media comprising: a) culturing a population of mammalian cells in a cell culture medium comprising 0.03 mg/L or less IGF-1; b) obtaining individual cells from the population of mammalian cells by single cell cloning; c) expanding and passaging the individual cells until recovered to 90% or greater viability and a doubling time less than 30 hours.

In certain embodiments, the cell culture media has no IGF-1.

In certain embodiments, the mammalian cells are Chinese Hamster Ovary (CHO) cells. In certain embodiments, the CHO cells are deficient in dihydrofolate reductase (DHFR) or a glutamine synthetase knock out (GSKO).

### BRIEF DESCRIPTION OF THE DRAWINGS

FIG. 1A-B depict A) gradual adaptation of GSKO host cell lines to a proprietary cell culture medium without Long R3 IGF-1 over an extended period of 110 population doubling levels (PDLs); and B) direct adaptation of GSKO host cells to a proprietary cell culture medium without Long R3 IGF-1 over a 1.5-month time period.

FIG. 2A-B illustrates doubling times of GSKO IGF⁻ adapted single cell cloned hosts compared to the GSKO controls. GSKO single cell cloned host cell lines were expanded and passaged until recovered to >90% and a doubling time of ˜24 hr.

FIG. 3 illustrates recovery graphs for 25 μM MSX recovered GSKO IGF⁻ adapted single cell cloned hosts post transfection with a monoclonal antibody. The IGF⁻ adapted cell lines in gray recover in a similar time period to the control designated by the black line.

FIGS. 4A-D: single cell cloned GSKO host cell lines transfected with a monoclonal antibody were inoculated at 1e6 or 3e6 cells/mL and assessed in a 15D fed batch production. The different shades of gray and black represent the parental host pools from which the single cell cloned hosts were derived. The shapes distinguish the individual cell lines. The transfected cell lines demonstrated variable levels of growth and productivity with several in the range of GSKO cell lines with IGF-1. A) Viable cell density graphs for GSKO single cell cloned transfected cell lines in a 15D Fed Batch (FB) production. B) Viability graphs for GSKO single cell cloned transfected cell lines in a 15D FB production. C) Titer graphs for GSKO single cell cloned transfected cell lines in a 15D FB production. D) Qp (volume specific productivity) graphs for GSKO single cell cloned transfected cell lines in a 15D FB production.

### DETAILED DESCRIPTION OF THE INVENTION

The present invention is based in part on the discovery that CHO host cells can be directly adapted to grow in IGF⁻ media (media lacking IGF-1) thereby obviating the need for the high levels of insulin like growth factor 1 (IGF-1) supplementation in the media. GSKO CHO hosts were directly adapted to a platform media without IGF-1 and single cell cloned to create robust host cell lines that retain or exceed the growth and productivity properties of the parental host cell lines grown in cell culture media that contains IGF-1. This invention arose, in part, from an effort to reduce the cost per gram of drug substance as IGF-1, a protein supplement that supports cell growth through signaling of the IGF-1R pathway, accounts for up to ˜30% of the media cost. Directly adapted CHO cells that can survive and grow without IGF-1 supplementation can reduce the high costs of IGF-1 in large-scale recombinant protein production. The IGF⁻ adapted host cell pools and subsequently single cell cloned IGF⁻ hosts have shown similar performance to the platform CHO hosts without the need for additional supplements.

The directly adapted cells disclosed herein show a proliferative rate that is the same or more than the proliferative rate of the original CHO cells. Also, the directly adapted cells show a production efficiency of a recombinant protein, which is the same or more than that of the original CHO cells. By using the directly adapted cell line of the present invention, biopharmaceuticals can be produced in a less expensive and more stable manner

The invention finds particular utility in the commercial production of proteins of interest in cell culture media lacking IGF-1. The methods described herein can employ IGF-1 free medium which is less expensive while maintaining similar production.

The cell lines (also referred to as “host cells”) used in the invention are directly adapted to grow in cell culture media in the absence of IGF-1, or having 0.03 mg/L or less IGF-1, and single clones are expanded, passaged and selected which have the desired properties. In certain embodiments, the cell lines also express a protein of commercial or scientific interest. Cell lines are typically derived from a lineage arising from a primary culture that can be maintained in culture for an unlimited time. Genetically engineering the cell line involves transfecting, transforming or transducing the cells with a recombinant polynucleotide molecule so as to cause the host cell to express a protein of interest. Methods and vectors for genetically engineering cells and/or cell lines to express, for example, a protein of interest, are well known to those of skill in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69.

### Definitions

While the terminology used in this application is standard within the art, definitions of certain terms are provided herein to assure clarity and definiteness in the meaning of the claims. Units, prefixes, and symbols may be denoted in their SI (International System of Units) accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990).

As used herein, the terms “a” and “an” mean one or more unless specifically indicated otherwise. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

All documents, or portions of documents, cited in this application, including but not limited to patents, patent applications, articles, books, and treatises, are hereby expressly incorporated by reference. What is described in an embodiment of the invention can be combined with other embodiments of the invention.

The present disclosure provides methods of expressing a “protein of interest”. A “protein of interest” includes naturally occurring proteins, recombinant proteins, and engineered proteins (e.g., proteins that do not occur in nature and which have been designed and/or created by humans). A protein of interest can, but need not be, a protein that is known or suspected to be therapeutically relevant.

As used herein, the terms “polypeptide” and “protein” (e.g., as used in the context of a protein of interest or a polypeptide of interest) are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell, or polypeptides and proteins can be produced by a genetically-engineered or recombinant cell. Polypeptides and proteins can comprise molecules having the amino acid sequence of a native protein, or molecules having deletions from, additions to, and/or substitutions of one or more amino acids of the native sequence.

The terms “polypeptide” and “protein” encompass molecules comprising only naturally occurring amino acids, as well as molecules that comprise non-naturally occurring amino acids. Examples of non-naturally occurring amino acids (which can be substituted for any naturally-occurring amino acid found in any sequence disclosed herein, as desired) include: 4-hydroxyproline, γ-carboxy glutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

A non-limiting list of examples of non-naturally occurring amino acids that can be inserted into a protein or polypeptide sequence or substituted for a wild-type residue in a protein or polypeptide sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

As used herein, the term “heterologous” used in connection with a nucleic acid means having a nucleic acid not naturally occurring within a host cell. This can include mutated sequences, e.g., sequences differing from the naturally occurring sequence. This can include sequences from other species. This can also include having a sequence at a different position in the genome than that naturally-occurring in the host cell. This generally does not include natural mutations that may occur in a host cell. A cell already containing a heterologous nucleic acid encoding a protein of interest, for example, by stable integration of an expression cassette, would be considered to contain a heterologous nucleic acid sequence. For clarity, a CHO cell or a derivative thereof (e.g., a DHFR- or GS knockout) having a nucleic acid encoding an antigen binding protein would be considered to have a heterologous nucleic acid.

The present disclosure contemplates both of the following: (1) host cells (e.g., CHO cells) that are first directly adapted to IGF⁻ media as described herein to create, for example, a master cell bank or working cell bank and then are further modified to incorporate a nucleic acid sequence encoding, for example, an antibody; and (2) cells, for example, master cell banks or working cell banks, that already have a nucleic acid encoding a heterologous protein of interest, e.g., an antibody, that are then directly adapted to IGF⁻ media as described herein.

As used herein, the term “bioreactor” means any vessel useful for the growth of a cell culture. The cell cultures of the instant disclosure can be grown in a bioreactor, which can be selected based on the application of a protein of interest that is produced by cells growing in the bioreactor. A bioreactor can be of any size so long as it is useful for the culturing of cells; typically, a bioreactor is sized appropriate to the volume of cell culture being grown inside of it. Typically, a bioreactor will be at least 1 liter and may be 2, 5, 10, 50, 100, 200, 250, 500, 1,000, 1500, 2000, 2,500, 5,000, 8,000, 10,000, 12,000 liters or more, or any volume in between. The internal conditions of the bioreactor, including, but not limited to pH and temperature, can be controlled during the culturing period. Those of ordinary skill in the art will be aware of, and will be able to select, suitable bioreactors for use in practicing the methods disclosed herein based on the relevant considerations.

As used herein, “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992) Mammalian cells may be cultured in suspension or while attached to a solid substrate. Fluidized bed bioreactors, hollow fiber bioreactors, roller bottles, shake flasks, or stirred tank bioreactors, with or without microcarriers, can be used. In one embodiment 500 L to 2000 L bioreactors are used. In one embodiment, 1000 L to 2000 L bioreactors are used.

The term “cell culture medium” (also called “culture medium,” “cell culture media,” “tissue culture media,”) refers to any nutrient solution used for growing cells, e g., animal or mammalian cells, and which generally provides at least one or more components from the following: an energy source (usually in the form of a carbohydrate such as glucose); one or more of all essential amino acids, and generally the twenty basic amino acids, plus cysteine; vitamins and/or other organic compounds typically required at low concentrations; lipids or free fatty acids; and trace elements, e.g., inorganic compounds or naturally occurring elements that are typically required at very low concentrations, usually in the micromolar range.

The nutrient solution may optionally be supplemented with additional optional components to optimize growth of cells, such as hormones and other growth factors, e.g., transferrin, epidermal growth factor, serum, and the like; salts, e.g., calcium, magnesium and phosphate, and buffers, e.g., HEPES; nucleosides and bases, e.g., adenosine, thymidine, hypoxanthine; and protein and tissue hydrolysates, e.g., hydrolyzed animal or plant protein (peptone or peptone mixtures, which can be obtained from animal byproducts, purified gelatin or plant material); antibiotics, e.g., gentamycin; cell protectants or surfactants such as Pluronic®F68 (also referred to as Lutrol® F68 and Kolliphor® P188; nonionic triblock composed of a central hydrophobic chain of polyoxypropylene (poly(propylene oxide)) flanked by two hydrophilic chains of polyoxyethylene (poly(ethylene oxide)); polyamines, e.g., putrescine, spermidine and spermine (see e.g., International Patent Application Publication No. WO 2008/154014) and pyruvate (see e.g. U.S. Pat. No. 8,053,238) depending on the requirements of the cells to be cultured and/or the desired cell culture parameters.

Cell culture media include those that are typically employed in and/or are known for use with any cell culture process, such as, but not limited to, batch, extended batch, fed-batch and/or perfusion or continuous culturing of cells.

A “base” (or batch) cell culture medium refers to a cell culture medium that is typically used to initiate a cell culture and is sufficiently complete to support the cell culture.

A “fed-batch culture” refers to a form of suspension culture and means a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (e.g., a cell-cycle inhibitory compound). A fed-batch culture is typically stopped at some point and the cells and/or components in the medium are harvested and optionally purified.

A “growth” cell culture medium refers to a cell culture medium that is typically used in cell cultures during a period of exponential growth, a “growth phase”, and is sufficiently complete to support the cell culture during this phase. A growth cell culture medium may also contain selection agents that confer resistance or survival to selectable markers incorporated into the host cell line. Such selection agents include, but are not limited to, geneticin (G418), neomycin, hygromycin B, puromycin, zeocin, methionine sulfoximine, methotrexate, glutamine-free cell culture medium, cell culture medium lacking glycine, hypoxanthine and thymidine, or thymidine alone.

A “perfusion” cell culture medium refers to a cell culture medium that is typically used in cell cultures that are maintained by perfusion or continuous culture methods and is sufficiently complete to support the cell culture during this process. Perfusion cell culture medium formulations may be richer or more concentrated than base cell culture medium formulations to accommodate the method used to remove the spent medium. Perfusion cell culture medium can be used during both the growth and production phases.

A “production” cell culture medium refers to a cell culture medium that is typically used in cell cultures during the transition when exponential growth is ending and protein production takes over, “transition” and/or “product” phases, and is sufficiently complete to maintain a desired cell density, viability and/or product titer during this phase.

Concentrated cell culture medium can contain some or all of the nutrients necessary to maintain the cell culture; in particular, concentrated medium can contain nutrients identified as or known to be consumed during the course of the production phase of the cell culture. Concentrated medium may be based on just about any cell culture media formulation. Such a concentrated feed medium can contain some or all the components of the cell culture medium at, for example, about 2×, 3×, 4×, 5×, 6×, 7×, 8×, 9×, 10×, 12×, 14×, 16×, 20×, 30×, 50×, 100×, 200×, 400×, 600×, 800×, or even about 1000× of their normal amount.

The components used to prepare cell culture medium may be completely milled into a powder medium formulation; partially milled with liquid supplements added to the cell culture medium as needed; or added in a completely liquid form to the cell culture.

Cell cultures can also be supplemented with independent concentrated feeds of particular nutrients which may be difficult to formulate or are quickly depleted in cell cultures. Such nutrients may be amino acids such as tyrosine, cysteine and/or cystine (see e.g., International Patent Application Publication No. WO2012/145682). For example, a concentrated solution of tyrosine can independently be fed to a cell culture grown in a cell culture medium containing tyrosine, such that the concentration of tyrosine in the cell culture does not exceed 8 mM. In another example, a concentrated solution of tyrosine and cystine is independently fed to the cell culture being grown in a cell culture medium lacking tyrosine, cystine or cysteine. The independent feeds can begin prior to or at the start of the production phase. The independent feeds can be accomplished by fed batch to the cell culture medium on the same or different days as the concentrated feed medium. The independent feeds can also be perfused on the same or different days as the perfused medium.

“Serum-free” applies to a cell culture medium that does not contain animal sera, such as fetal bovine serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), MCDB 302 (Sigma Aldrich Corp., St. Louis, Mo.), among others. Serum-free versions of such culture media are also available. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. Customized cell culture media can also be used.

“Cell density” refers to the number of cells in a given volume of culture medium. “Viable cell density” refers to the number of live cells in a given volume of culture medium, as determined by standard viability assays (such as trypan blue dye exclusion method).

“Cell viability” means the ability of cells in culture to survive under a given set of culture conditions or experimental variations. The term also refers to that portion of cells which are alive at a particular time in relation to the total number of cells, living and dead, in the culture at that time.

“Growth-arrest”, which may also be referred to as “cell growth-arrest”, is the point where cells stop increasing in number or when the cell cycle no longer progresses. Growth-arrest can be monitored by determining the viable cell density of a cell culture. Some cells in a growth-arrested state may increase in size but not number, so the packed cell volume of a growth-arrested culture may increase. Growth-arrest can be reversed to some extent, if the cells are not in declining health, by reversing the conditions that lead to growth arrest.

“Packed cell volume” (PCV), also referred to as “percent packed cell volume” (% PCV), is the ratio of the volume occupied by the cells, to the total volume of cell culture, expressed as a percentage (see Stettler et al., 2006, Biotechnol Bioeng. December 20:95(6):1228-33). Packed cell volume is a function of cell density and cell diameter; increases in packed cell volume could arise from increases in either cell density or cell diameter or both. Packed cell volume is a measure of the solid content in the cell culture. Solids are removed during harvest and downstream purification. ore solids mean more effort to separate the solid material from the desired product during harvest and downstream purification steps. Also, the desired product can become trapped in the solids and lost during the harvest process, resulting in a decreased product yield. Since host cells vary in size and cell cultures also contain dead and dying cells and other cellular debris, packed cell volume is a more accurate way to describe the solid content within a cell culture than cell density or viable cell density. For example, a 2000 L culture having a cell density of 50×10⁶ cells/ml would have vastly different packed cell volumes depending on the size of the cells. In addition, some cells, when in a growth-arrested state, will increase in size, so the packed cell volume prior to growth-arrest and post growth-arrest will likely be different, due to increase in biomass as a result to cell size increase.

“Titer” means the total amount of a polypeptide or protein of interest (which may be a naturally occurring or recombinant protein of interest) produced by a cell culture in a given amount of medium volume. Titer can be expressed in units of milligrams or micrograms of polypeptide or protein per milliliter (or other measure of volume) of medium. “Cumulative titer” is the titer produced by the cells during the course of the culture, and can be determined, for example, by measuring daily titers and using those values to calculate the cumulative titer.

As used herein, the term “host cell” is understood to include a cell that has been genetically engineered to express a polypeptide of interest. Genetically engineering a cell involves transfecting, transforming or transducing the cell with a nucleic acid encoding a recombinant polynucleotide molecule (a “gene of interest”), and/or otherwise altering (e.g., by homologous recombination and gene activation or fusion of a recombinant cell with a non-recombinant cell) so as to cause the host cell to express a desired recombinant polypeptide. Methods and vectors for genetically engineering cells and/or cell lines to express a polypeptide of interest are well known to those of skill in the art; for example, various techniques are illustrated in Current Protocols in Molecular Biology. Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69. The term includes the progeny of the parent cell, whether or not the progeny is identical in morphology or in genetic makeup to the original parent cell, so long as the gene of interest is present. A cell culture can comprise one or more host cells.

IGF-1 is a polypeptide protein hormone similar in molecular structure to insulin. In addition, IGF-1 plays an important role in growth and anabolism of adult mammals.

IGF-1R has a binding site for ATP, which is used to provide the phosphates for autophosphorylation. The structures of the autophosphorylation complexes of tyrosine residues 1165 and 1166 have been identified within crystals of the IGF1R kinase domain. See Xu et al., 2015, Science Signaling 8(405):rs13. In response to ligand binding, the a chains induce the tyrosine autophosphorylation of the β chains This event triggers a cascade of intracellular signaling that, while cell type-specific, often promotes cell survival and cell proliferation. See Jones et al., 1995, Endocrine Reviews 16(1):3-34 and LeRoith et al., 1995, Endocrine Reviews 16(2):143-63. It is this effect on cell proliferation that makes the supplementation of cell culture media with IGF-1 commonplace in large scale production of recombinant proteins.

IGF-1 is commercially available and is typically used as a supplement for cell culture media at a concentration of about 0.1 mg/L. There are at least three commercially available forms of IGF-1 which can be included in cell culture media, including native IGF-1 (70 amino acids, 7.6 kDa, available from, for example, R&D Systems) Long R3 IGF-1 (83 amino acids, 9.1 kDa, available from, for example, Millipore Sigma and Repligen) and Short™ AE-IGF-1 (72 amino acids, 7.9 kDa, available from, for example, CellRx).

### Method for Direct Adaption of Mammalian Cells to IGF− Media

By directly adapting a mammalian cell to IGF⁻ media (media lacking IGF-1), it has been discovered that IGF-1 can be reduced or omitted in large scale recombinant protein manufacturing while retaining similar growth rates and productivity. Directly adapting a mammalian cell to IGF− media means using a cell culture that has been grown or previously had been grown (and subsequently frozen) in cell culture media containing IGF-1, including IGF-1 available in serum, and culturing these cells directly into cell culture media lacking IGF-1. In direct adaptation, the cells are only adapted to a single cell culture media having a concentration of IGF-1, which can include no IGF-1. This is contrasted with a gradual adaptation which involves serially reducing the amount of IGF-1 present in the cell culture media and allowing the cells to recover at each step of reducing the IGF-1 concentration.

The present disclosure provides a method for directly adapting a mammalian cell to IGF− media comprising: a) culturing a population of mammalian cells in a cell culture medium comprising 0.03 mg/L or less IGF-1; b) obtaining individual cells from the population of mammalian cells by single cell cloning; and c) expanding and passaging the individual cells until recovered to 90% or greater and a doubling time less than 30 hours. The best clones are selected based on characteristics such as viability, growth and transfectability.

Cell culture media lacking IGF-1 generally means that the cell culture media contains a reduced level of IGF-1 compared to standard cell culture conditions. For example, the cell culture media for direct adaptation (sometimes referred to herein as a first cell culture media) can contain 0.03 mg/L or less, 0.02 mg/L or less, 0.01 mg/L or less, or no IGF-1. IGF⁻ media refers to cell culture media lacking IGF-1.

In the methods disclosed herein, any mammalian cell line can be used. A wide variety of mammalian cell lines suitable for growth in culture are available from the American Type Culture Collection (Manassas, Va.) and commercial vendors. Examples of cell lines commonly used in the industry include monkey kidney CV1 line transformed by SV40 (COS-7, ATCC CRL 1651); human embryonic kidney line (293 or 293 cells subcloned for growth in suspension culture, (Graham et al, 1977, J. Gen Virol. 36:59); baby hamster kidney cells (BHK, ATCC CCL 10); mouse Sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., 1982, Annals N.Y Acad. Sci. 383:44-68); MRC 5 cells or FS4 cells; mammalian myeloma cells, and a number of other cell lines and Chinese hamster ovary (CHO) cells.

Large-scale production of proteins for commercial applications is typically carried out in suspension culture. Therefore, mammalian host cells used to generate the recombinant mammalian cells described herein can, but need not be, adapted to growth in suspension culture. A variety of host cells adapted to growth in suspension culture are known, including mouse myeloma NS0 cells and CHO cells from CHO-S, DG44, and DXB11 cell lines. Other suitable cell lines include mouse myeloma SP2/0 cells, baby hamster kidney BHK-21 cells, human PER.C6® cells, human embryonic kidney HEK-293 cells, and cell lines derived or engineered from any of the cell lines disclosed herein.

CHO cells are widely used to produce complex recombinant proteins, including CHOK1 cells (ATCC CCL61). The dihydrofolate reductase (DHFR)-deficient mutant cell lines (Urlaub et al., 1980, Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J., 1990, Meth Enzymol 185:537-566). Also included are the glutamine synthase (GS)-knockout CHOK1SV cell lines, making use of glutamine synthetase (GS)-based methionine sulfoximine (MSX) selection. Other suitable CHO host cells could include, but are not limited to the following (ECACC accession numbers in brackets): CHO (85050302), CHO (PROTEIN FREE) (00102307), CHO-K1 (85051005), CHO-K1/SF (93061607), CHO/DHFR-(94060607), CHO/DHFR-AC-free (05011002), RR-CHOKI (92052129).

A cell culture of a mammalian cell line in a cell culture media containing its usual and preferred components is used for direct adaptation. Typically, this cell culture media includes serum with IGF-1. The cells are preferably cultured, and optionally frozen, while in an exponential growth phase.

The mammalian cells are passaged in a cell culture media lacking IGF-1. In certain embodiments, the IGF-1 concentration is 0.03 mg/L or less. In certain embodiments, the IGF-1 concentration is 0 mg/L. Preferably, single cells are cloned, for example on a Berkley Lights (BLI) Beacon Instrument. The cells are expanded and passaged until they are adapted to the IGF⁻ media, e.g., they have a viability of 90% or greater and they are able to proliferate at a normal growth rate, e.g., a doubling time of 30 hours or less.

The methods and cell lines described herein employing IGF⁻ direct adaptation allow for the reduction of the amounts of IGF-1 in the cell culture media used for manufacturing a protein of interest. Typically, the concentration of IGF-1 is cell culture media is 0.1 mg/L. In the methods disclosed herein, the concentration of IGF-1 in the cell culture media can be reduced to less than equal to 0.05, 0.04, 0.03, 0.02, or 0.01 mg/L. In certain embodiments, no IGF-1 is need in the cell culture media, i.e., the concentration of IGF-1 is the cell culture media is 0 mg/L.

In the methods described herein, the cells have a growth rate comparable to a cell of the same lineage without IGF⁻ adaptation. In certain embodiments, the growth rate is 0.015-0.04 l/hr for the first 5 days of production. In certain embodiments, the growth rate is 0.022-0.025 l/hr in a seed train. In certain embodiments, the cells have a doubling time of 20-30 or 23-35 hours.

In the methods described herein, the cells produce a recombinant protein of interest at a titer comparable to a cell of the same lineage without having been adapted to cell culture media without IGF-1. In certain embodiments, the titer of the protein of interest is at least 50 mg/L, 100 mg/L, 150 mg/L, 200 mg/L, 250 mg/L, 300 mg/L, 350 mg/L, 400 mg/L, 450 mg/L, 500 mg/L, 550 mg/L, or 600 mg/L after day 10 of a culture.

### Generation of Mammalian Host Cells Expressing a Protein of Interest

Expression of a protein of interest in a cell can be achieved by well-known methods, either transiently or by stable expression (Davis et al., Basic Methods in Molecular Biology, 2^{nd} ed., Appleton & Lange, Norwalk, Conn., 1994; Sambrook et al., Molecular Cloning: A Laboratory Manual, 3rd ed., Cold Spring Harbor Laboratory Press, Cold Spring Harbor, N.Y., 2001).

Methods for stable integration are well known in the art. Briefly, stable integration is commonly achieved by transiently introducing a heterologous polynucleotide or a vector containing the heterologous polynucleotide into the host cell, which facilitates the stable integration of said heterologous polynucleotide into the cell genome. Typically, the heterologous polynucleotide is flanked by homology arms, i.e., sequences homologous to the region upstream and downstream to the integration site. Before their introduction into the mammalian host cell, circular vectors may be linearized to facilitate integration into the cell genome. Methods for the introduction of vectors into cells are well known in the art and include transfection with biological methods, such as viral delivery, with chemical methods, such as using cationic polymers, calcium phosphate, cationic lipids or cationic amino acids; with physical methods, such as electroporation or microinjection; or with mixed approaches, such as protoplast fusion.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

A specific method of stable integration uses recombinase mediated cassette exchange (RMCE; Bode and Baer, 2001, Curr Opin Biotechnol. 12:473-80, and Bode et al., 2000, Biol. Chem. 381:801-813) for site-specific integration in the genome (also termed “targeted integration”). Site- specific recombinases such as Flp and Cre mediate recombination between two copies of their target sequence termed FRT and loxP, respectively. The use of two incompatible target sequences, for example FRT in combination with F3 (Schlake and Bode, 1994, Biochemistry, 33:12746-51) as well as inverted recognition target sites (Feng et al., 1999, J. Mol. Biol. 292:779-85) allows the insertion of DNA segments into a predefined chromosomal locus carrying target sequences in a similar configuration. See also EP Patent No. EP1781796B1 and EP Patent Application Publication No. EP2789691A1.

Insertion of RMCE into a specific site in the genome can be mediated by nucleases (e.g., zinc finger protein (ZFP), transcription activator-like effector nuclease (TALEN), clustered regularly interspaced short palindromic repeats (CRISPR)/CRISPR-associated protein 9 (Cas9)) that can be engineered to create single- and double-stranded breaks (SSBs/DSBs) in the genome. There are two major and distinct pathways to repair DSBs—homologous recombination and non-homologous end joining (NHEJ). Homologous recombination requires the presence of a homologous sequence as a template (e.g., “donor” containing RMCE) to guide the cellular repair process and the results of the repair are error-free and predictable. In the absence of a template (or “donor”) sequence for homologous recombination, the cell typically attempts to repair the DSB via the unpredictable and error-prone process of non-homologous end joining (NHEJ).

A vector may be any molecule or entity (e.g., nucleic acid, plasmid, bacteriophage, transposon, cosmid, chromosome, virus, virus capsid, virion, naked DNA, complexed DNA and the like) suitable for use to transfer and/or transport protein encoding information into a host cell and/or to a specific location and/or compartment within a host cell. Vectors can include viral and non-viral vectors, non-episomal mammalian vectors. Vectors are often referred to as expression vectors, for example, recombinant expression vectors and cloning vectors. The vector may be introduced into a host cell to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art.

Vectors are useful for transformation of a host cell and contain nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

Vectors may be selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964). Suitable expression vectors are known in the art and are also commercially available.

Typically, vectors used in host cells will contain sequences for plasmid maintenance and for cloning and expression of exogenous nucleotide sequences. Such sequences will typically include one or more of the following nucleotide sequences: a promoter, one or more enhancer sequences, an origin of replication, transcriptional and translational control sequences, a transcriptional termination sequence, a complete intron sequence containing a donor and acceptor splice site, various pre- or pro-sequences to improve glycosylation or yield, a native or heterologous signal sequence (leader sequence or signal peptide) for polypeptide secretion, a ribosome binding site, a polyadenylation sequence, internal ribosome entry site (IRES) sequences, an expression augmenting sequence element (EASE), tripartite leader (TPA) and VA gene RNAs from Adenovirus 2, a polylinker region for inserting the polynucleotide encoding the polypeptide to be expressed, and a selectable marker element. Vectors may be constructed from a starting vector such as a commercially available vector, additional elements may be individually obtained and ligated into the vector. Methods used for obtaining each of the components are well known to one skilled in the art.

Vector components may be homologous (i.e., from the same species and/or strain as the host cell), heterologous (e.g., from a species other than the host cell species or strain), hybrid (i.e., a combination of flanking sequences from more than one source), synthetic or native. The sequences of components useful in the vectors may be obtained by methods well known in the art, such as those previously identified by mapping and/or by restriction endonuclease. In addition, they can be obtained by polymerase chain reaction (PCR) and/or by screening a genomic library with suitable probes.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

An origin of replication aids in the amplification of the vector in a host cell. They may be included as part of commercially available prokaryotic vectors and may also be chemically synthesized based on a known sequence and ligated into the vector. Various viral origins (e.g., SV40, polyoma, adenovirus, vesicular stomatitus virus (VSV), or papillomaviruses such as HPV or BPV) are useful for cloning vectors in mammalian cells.

Transcriptional and translational control sequences for mammalian host cell expression vectors can be excised from viral genomes. Commonly used promoter and enhancer sequences are derived from polyoma virus, adenovirus 2, simian virus 40 (SV40), and human cytomegalovirus (CMV). For example, the human CMV promoter/enhancer of immediate early gene 1 may be used. See e.g. Patterson et al., 1994, Applied Microbiol. Biotechnol. 40:691-98. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., 1978, Nature 273:113; Kaufman, 1990, Meth. in Enzymol. 185:487-511). Smaller or larger SV40 fragments can also be used, provided the approximately 250 bp sequence extending from the Hind III site toward the BglI site located in the SV40 viral origin of replication site is included.

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis known to those of skill in the art.

A selectable marker gene encoding a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include glutamine synthase (GS), dihydrofolate reductase (DHFR), and promoterless thymidine kinase genes Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes a protein of interest. As a result, increased quantities of a polypeptide of interest are synthesized from the amplified DNA.

In some cases, such as where glycosylation is desired in a eukaryotic host cell expression system, one may manipulate the various pre- or pro-sequences to improve glycosylation or yield. For example, one may alter the peptidase cleavage site of a particular signal peptide, or add prosequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide if the enzyme cuts at such area within the mature polypeptide.

Expression and cloning will typically contain a promoter that is recognized by the host organism and operably linked to the molecule encoding a protein of interest. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known.

Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thomsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78:1444-1445); glyceraldehyde-3-phosphate dehydrogenase (GAPDH); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315:115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7:1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5:1639-1648; Hammer et al., 1987, Science 253:53-58); the alpha 1-antitrypsin gene control region that is active in liver (Kelsey et al., 1987, Genes and Devel. 1:161-171); the beta-globin gene control region that is active in myeloid cells (Mogram et al., 1985, Nature 315:338-340; Kollias et al., 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234:1372-1378).

An enhancer sequence may be inserted into the vector to increase transcription by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter.

A sequence encoding an appropriate native or heterologous signal sequence (leader sequence or signal peptide) can be incorporated into an expression vector, to promote extracellular secretion of the protein of interest. The choice of signal peptide or leader depends on the type of host cells in which the protein of interest to be produced, and a heterologous signal sequence can replace the native signal sequence. Examples of signal peptides that are functional in mammalian host cells include the following: the signal sequence for interleukin-7 described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

Additional control sequences shown to improve expression of heterologous genes from mammalian expression vectors include such elements as the expression augmenting sequence element (EASE) derived from CHO cells (Morris et al., in Animal Cell Technology, pp. 529-534 (1997); U.S. Pat. Nos. 6,312,951 B1, 6,027,915, and 6,309,841 B1) and the tripartite leader (TPL) and VA gene RNAs from Adenovirus 2 (Gingeras et al., 1982, J. Biol. Chem. 257:13475-13491). The internal ribosome entry site (IRES) sequences of viral origin allows bicistronic mRNAs to be translated efficiently (Oh and Sarnow, 1993, Current Opinion in Genetics and Development 3:295-300; Ramesh et al., 1996, Nucleic Acids Research 24:2697-2700).

Following construction, one or more vectors may be inserted into a suitable cell for amplification and/or polypeptide expression. The transformation of an expression vector into a selected cell may be accomplished by well-known methods including transfection, infection, calcium phosphate co-precipitation, electroporation, nucleofection, microinjection, DEAE-dextran mediated transfection, cationic lipids mediated delivery, liposome mediated transfection, microprojectile bombardment, receptor-mediated gene delivery, delivery mediated by polylysine, histone, chitosan, and peptides. The method selected will in part be a function of the type of host cell to be used. These methods and other suitable methods are well known to the skilled artisan and are set forth in manuals and other technical publications, for example, in Sambrook et al., Molecular Cloning: A Laboratory Manual, 3rd ed., Cold Spring Harbor Laboratory Press, Cold Spring Harbor, N.Y. (2001).

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197.

The term “transduction” refers to the process whereby foreign DNA is introduced into a cell via viral vector. See Jones et al., (1998). Genetics: principles and analysis. Boston: Jones & Bartlett Publ.

### Description of Cell Culture Process

In the methods described herein, using reduced amounts of IGF-1 or no IGF-1 can be performed at any or all stages of a production run. Sometimes the cell culture media used for production is referred to herein as a second cell culture media. This second cell culture media does not have to have the same concentration of IGF-1 as the first cell culture media. The second cell culture media can have an IGF-1 concentration of 0.05 mg/L or less, 0.03 mg/L or less, 0.02 mg/L or less, 0.01 mg/L or less or no IGF-1. For example, IGF-1 can be reduced to 0.03 mg/L or less at a seed scale, at a production scale (N) or anywhere in between (e.g., N-1, N-2, etc.). At the production scale, IGF-1 can be reduced in the initial cell culture media and/or the perfusion media or fed-batch feed media, as appropriate.

The disclosed methods are applicable to adherent culture or suspension cultures grown in stirred tank reactors (including traditional batch and fed-batch cell cultures, which may but need not comprise a spin filter), perfusion systems (including alternating tangential flow (“ATF”) cultures, acoustic perfusion systems, depth filter perfusion systems, and other systems), hollow fiber bioreactors (HFB, which in some cases may be employed in perfusion processes) as well as various other cell culture methods (see, e.g., Tao et al., 2003, Biotechnol. Bioeng. 82:751-65; Kuystermans & Al-Rubeai, (2011) “Bioreactor Systems for Producing Antibody from Mammalian Cells” in Antibody Expression and Production, Cell Engineering 7:25-52, Al-Rubeai (ed) Springer; Catapano et al., (2009) “Bioreactor Design and Scale-Up” in Cell and Tissue Reaction Engineering: Principles and Practice, Eibl et al. (eds) Springer-Verlag, incorporated herein by reference in their entireties).

During recombinant protein production it is desirable to have a controlled system where cells are grown to a desired density and then the physiological state of the cells is switched to a growth-arrested, high productivity state where the cells use energy and substrates to produce the recombinant protein of interest instead of making more cells. Various methods for accomplishing this goal exist, and include temperature shifts and amino acid starvation, as well as use of a cell-cycle inhibitor or other molecule that can arrest cell growth without causing cell death.

The production of a recombinant protein begins with establishing a mammalian cell production culture of cells that express the protein, in a culture plate, flask, tube, bioreactor or other suitable vessel. Smaller production bioreactors are typically used, in one embodiment the bioreactors are 500 L to 2000 L. In another embodiment, 1000 L-2000 L bioreactors are used. The seed cell density used to inoculate the bioreactor can have a positive impact on the level of recombinant protein produced. In one embodiment the bioreactor is inoculated with at least 0.5×10⁶ up to and beyond 3.0×10⁶ viable cells/mL in a serum-free culture medium. In another embodiment the inoculation is 1.0×10⁶ viable cells/mL.

The mammalian cells then undergo an exponential growth phase. The cell culture can be maintained without supplemental feeding until a desired cell density is achieved. In one embodiment the cell culture is maintained for up to three days with or without supplemental feeding. In another embodiment the culture can be inoculated at a desired cell density to begin the production phase without a brief growth phase. In any of the embodiments herein the switch from the growth phase to production phase can also be initiated by any of the afore-mentioned methods.

At the transition between the growth phase and the production phase, and during the production phase, the percent packed cell volume (% PCV) can be equal to or less than 35%. For example, the desired packed cell volume maintained during the production phase is equal to or less than 35%, equal to or less than 30%, equal to or less than 20%, equal to or less than 15%, or equal to or less than 10%.

The desired viable cell density at the transition between the growth and production phases and maintained during the production phase can be various depending on the projects. It can be decided based on the equivalent packed cell volume from the historical data. For example, the viable cell density can be at least about 10×10⁶ viable cells/mL to 80×10⁶ viable cells/mL, at least about 10×10⁶ viable cells/mL to 70×10⁶ viable cells/mL, at least about 10×10⁶ viable cells/mL to 60×10⁶ viable cells/mL, at least about 10×10⁶ viable cells/mL to 50×10⁶ viable cells/mL, at least about 10×10⁶ viable cells/mL to 40×10⁶ viable cells/mL, at least about 10×10⁶ viable cells/mL to 30×10⁶ viable cells/mL, at least about 10×10⁶ viable cells/mL to 20×10⁶ viable cells/mL, at least about 20×10⁶ viable cells/mL to 30×10⁶ viable cells/mL, at least about 20×10⁶ viable cells/mL to at least about 25×10⁶ viable cells/mL, or at least about 20×10⁶ viable cells/mL.

Lower packed cell volume during the production phase helps mitigate dissolved oxygen sparging problems that can hinder higher cell density perfusion cultures. The lower packed cell volume also allows for a smaller media volume which allows for the use of smaller media storage vessels and can be combined with slower flow rates. Lower packed cell volume also has less impact on harvest and downstream processing, compared to higher cell biomass cultures. All of which reduces the costs associated with manufacturing recombinant protein therapeutics.

Three methods are typically used in commercial processes for the production of recombinant proteins by mammalian cell culture: batch culture, fed-batch culture, and perfusion culture. Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method, however viable cell density is limited by the nutrient availability and once the cells are at maximum density, the culture declines and production decreases. There is no ability to extend a production phase because the accumulation of waste products and nutrient depletion rapidly lead to culture decline, (typically around 3 to 7 days).

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/ml, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed batch cultures have the potential to achieve higher product titers compared to batch cultures. Typically, a batch method is used during the growth phase and a fed-batch method used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, about 10 to 21 days. Fed-batch cultures are discontinuous, and harvest typically occurs when metabolic by-product levels or culture viability reach predetermined levels. When compared to a batch culture, in which no feeding occurs, a fed batch culture can produce greater amounts of recombinant protein. See e.g. U.S. Pat. No. 5,672,502.

Perfusion methods offer potential improvement over the batch and fed-batch methods by adding fresh media and simultaneously removing spent media. Typical large scale commercial cell culture strategies strive to reach high cell densities, 60-90(+)×10⁶ cells/mL where almost a third to over one-half of the reactor volume is biomass. With perfusion culture, extreme cell densities of >1×10⁸ cells/mL have been achieved and even higher densities are predicted. Typical perfusion cultures begin with a batch culture start-up lasting for a day or two followed by continuous, step-wise and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Perfusion flow rates of a fraction of a working volume per day up to many multiple working volumes per day have been reported.

An advantage of the perfusion process is that the production culture can be maintained for longer periods than batch or fed-batch culture methods. However, increased media preparation, use, storage and disposal are necessary to support a long-term perfusion culture, particularly those with high cell densities, which also need even more nutrients, and all of this drives the production costs even higher, compared to batch and fed batch methods. In addition, higher cell densities can cause problems during production, such as maintaining dissolved oxygen levels and problems with increased gassing including supplying more oxygen and removing more carbon dioxide, which would result in more foaming and the need for alterations to antifoam strategies; as well as during harvest and downstream processing where the efforts required to remove the excessive cell material can result in loss of product, negating the benefit of increased titer due to increased cell mass.

Also provided is a large-scale cell culture strategy that combines fed batch feeding during the growth phase followed by continuous perfusion during the production phase. The method targets a production phase where the cell culture is maintained at a packed cell volume of less than or equal to 35%.

In one embodiment, a fed-batch culture with bolus feeds is used to maintain a cell culture during the growth phase. Perfusion feeding can then be used during a production phase. In one embodiment, perfusion begins when the cells have reached a production phase. In another embodiment, perfusion begins on or about day 3 to on or about day 9 of the cell culture. In another embodiment perfusion begins on or about day 5 to on or about day 7 of the cell culture.

Using bolus feeding during the growth phase allows the cells to transition into the production phase, resulting in less dependence on a temperature shift as a means of initiating and controlling the production phase, however a temperature shift of about 36° C. to about 31° C. can take place between the growth phase and production phase. In one embodiment the shift is from 36° C. to 32° C.

As described herein, the bioreactor can be inoculated with at least 0.5×10⁶ up to and beyond 3.0×10⁶ viable cells/mL in a serum-free culture medium, for example 1.0×10⁶ viable cells/mL.

Perfusion culture is one in which the cell culture receives fresh perfusion feed medium while simultaneously removing spent medium. Perfusion can be continuous, stepwise, intermittent, or a combination of any or all of any of these. Perfusion rates can be less than a working volume to many working volumes per day. The cells are retained in the culture and the spent medium that is removed is substantially free of cells or has significantly fewer cells than the culture. Recombinant proteins expressed by the cell culture can also be retained in the culture. Perfusion can be accomplished by a number of means including centrifugation, sedimentation, or filtration, See e.g. Voisard et al., 2003, Biotechnology and Bioengineering 82:751-65. An example of a filtration method is alternating tangential flow filtration. Alternating tangential flow is maintained by pumping medium through hollow-fiber filter modules. See e.g. U.S. Pat. No. 6,544,424; Furey, 2002, Gen. Eng. News. 22 (7):62-63.

“Perfusion flow rate” is the amount of media that is passed through (added and removed) from a bioreactor, typically expressed as some portion or multiple of the working volume, in a given time. “Working volume” refers to the amount of bioreactor volume used for cell culture. In one embodiment the perfusion flow rate is one working volume or less per day. Perfusion feed medium can be formulated to maximize perfusion nutrient concentration to minimize perfusion rate.

Cell cultures can be supplemented with concentrated feed medium containing components, such as nutrients and amino acids, which are consumed during the course of the production phase of the cell culture.

Concentrated feed medium may be based on just about any cell culture media formulation. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

The method according to the present invention may be used to improve the production of recombinant proteins in multiple phase culture processes. In a multiple stage process, cells are cultured in two or more distinct phases. For example, cells may be cultured first in one or more growth phases, under environmental conditions that maximize cell proliferation and viability, then transferred to a production phase, under conditions that maximize protein production. In a commercial process for production of a protein by mammalian cells, there are commonly multiple, for example, at least about 2, 3, 4, 5, 6, 7, 8, 9, or 10 growth phases that occur in different culture vessels preceding a final production culture.

The growth and production phases may be preceded by, or separated by, one or more transition phases. In multiple phase processes, the method according to the present invention can be employed at least during the growth and production phase of the final production phase of a commercial cell culture, although it may also be employed in a preceding growth phase. A production phase can be conducted at large scale. A large-scale process can be conducted in a volume of at least about 100, 500, 1000, 2000, 3000, 5000, 7000, 8000, 10,000, 15,000, 20,000 liters. In one embodiment production is conducted in 500 L, 1000 L and/or 2000 L bioreactors.

A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift. The cell cultures can be maintained for days or even weeks while the cells produce the desired protein(s).

Samples from the cell culture can be monitored and evaluated using any of the analytical techniques known in the art. A variety of parameters including recombinant protein and medium quality and characteristics can be monitored for the duration of the culture. Samples can be taken and monitored intermittently at a desirable frequency, including continuous monitoring, real time or near real time.

Typically, the cell cultures that precede the final production culture (N-x to N-1) are used to generate the seed cells that will be used to inoculate the production bioreactor, the N-1 culture. The seed cell density can have a positive impact on the level of recombinant protein produced. Product levels tend to increase with increasing seed density. Improvement in titer is tied not only to higher seed density, but is likely to be influenced by the metabolic and cell cycle state of the cells that are placed into production.

Seed cells can be produced by any culture method. One such method is a perfusion culture using alternating tangential flow filtration. An N-1 bioreactor can be run using alternating tangential flow filtration to provide cells at high density to inoculate a production bioreactor. The N-1 stage may be used to grow cells to densities of >90×10⁶ cells/mL. The N-1 bioreactor can be used to generate bolus seed cultures or can be used as a rolling seed stock culture that could be maintained to seed multiple production bioreactors at high seed cell density. The duration of the growth stage of production can range from 7 to 14 days and can be designed so as to maintain cells in exponential growth prior to inoculation of the production bioreactor. Perfusion rates, medium formulation and timing are optimized to grow cells and deliver them to the production bioreactor in a state that is most conducive to optimizing their production. Seed cell densities of >15×10⁶ cells/mL can be achieved for seeding production bioreactors. Higher seed cell densities at inoculation can decrease or even eliminate the time needed to reach a desired production density.

In certain embodiments, the mammalian host cells and methods of the present disclosure can be used to generate high yield of a protein of interest. High yield, or high volumetric productivity, to the ability of cells to produce high levels of a protein of interest. The particular yield will depend on the protein of interest and can be at least 0.05 g/L, at least 0.1 g/L, at least 0.15 g/L, at least 0.2 g/L, at least 0.25 g/L, at least 0.3 g/L, at least 0.35 g/L, at least 0.4 g/L, at least 0.45 g/L, at least 0.5 g/L, at least 0.6 g/L, at least 0.7 g/L, at least 0.8 g/L, at least 0.9 g/L, at least 1 g/L, at least 1.5 g/L, at least 2 g/L, or more, in a 10-day culture grown in fed batch or perfusion conditions, using a feed medium suitable for the mammalian host cell and containing amino acids, vitamins, or trace elements, while containing reduced amounts or lacking IGF-1. In specific embodiments, the host cells and methods of the present disclosure express a protein of interest and are capable of producing at least 0.5 g/L, at least 0.6 g/L, at least 0.7 g/L, at least 0.8 g/L, at least 0.9 g/L, at least 1 g/L, at least 1.5 g/L, at least 2 g/L, or more, preferably up to about 3 g/L, 4 g/L, 5 g/L or 10 g/L when grown under the culture conditions described above.

Yield can also be measured in terms of the specific productivity of a cell line, determined based on the amount of protein produced per cell per day (expressed as pg/cell/day) Mammalian host cells of the present disclosure are capable of producing at least 1 pg/cell/day, at least 2 pg/cell/day, at least 3 pg/cell/day, at least 4 pg/cell/ day, at least 5 pg/cell/day, at least 6 pg/cell/day, at least 7 pg/cell/day, at least 8 pg/cell/day, at least 9 pg/cell/day, at least 10 pg/cell/day, at least 11 pg/cell/day, at least 12 pg/cell/day, at least 13 pg/cell/day, at least 14 pg/cell/day, at least 15 pg/cell/day, at least 20 pg/cell/day, at least 25 pg/cell/day, or more, preferably up to 50 pg/cell/day in a 10-day culture grown in fed batch or perfusion conditions, using a feed medium suitable for the mammalian host cell and containing amino acids, vitamins, or trace elements, while containing reduced amounts or lacking IGF-1. In specific embodiments, mammalian host cells of the present disclosure express an protein of interest and have a specific productivity of at least 10 pg/cell/day, at least 11 pg/cell/day, at least 12 pg/cell/day, at least 13 pg/cell/day, at least 14 pg/cell/day, at least 15 pg/cell/day, at least 20 pg/cell/day, at least 25 pg/cell/day, or more, preferably up to 50 pg/cell/day under the culture conditions described above.

The methods described herein can be used to culture cells that express a protein of interest. The expressed protein may be secreted into the culture medium from which they can be recovered and/or collected. In addition, the proteins can be purified, or partially purified, from such culture or component (e.g., from culture medium) using known processes and products available from commercial vendors. The purified proteins can then be “formulated”, meaning buffer exchanged, sterilized, bulk-packaged, and/or packaged for a final user. Suitable formulations for pharmaceutical compositions include those described in Remington's Pharmaceutical Sciences, 18th ed. 1995, Mack Publishing Company, Easton, Pa.

### Proteins of Interest

Polypeptides and proteins of interest can be of scientific or commercial interest, including protein-based therapeutics. Proteins of interest include, among other things, secreted proteins, non-secreted proteins, intracellular proteins or membrane-bound proteins. Polypeptides and proteins of interest can be produced by recombinant animal cell lines using cell culture methods and may be referred to as “recombinant proteins”. The expressed protein(s) may be produced intracellularly or secreted into the culture medium from which it can be recovered and/or collected. The term “isolated protein” or “isolated recombinant protein” refers to a polypeptide or protein of interest, that is purified away from proteins or polypeptides or other contaminants that would interfere with its therapeutic, diagnostic, prophylactic, research or other use. Proteins of interest include proteins that exert a therapeutic effect by binding a target, particularly a target among those listed below, including targets derived therefrom, targets related thereto, and modifications thereof.

Proteins of interest include “antigen-binding proteins”. Antigen-binding protein refers to proteins or polypeptides that comprise an antigen-binding region or antigen-binding portion that has affinity for another molecule to which it binds (antigen). Antigen-binding proteins encompass antibodies, peptibodies, antibody fragments, antibody derivatives, antibody analogs, fusion proteins (including single-chain variable fragments (scFvs), double-chain (divalent) scFvs, and IgGscFv (see, e.g., Orcutt et al., 2010, Protein Eng Des Sel 23:221-228), hetero-IgG (see, e.g., Liu et al., 2015, J Biol Chem 290:7535-7562), muteins, and XmAb® (Xencor, Inc., Monrovia, Calif.). Examples of antigen binding proteins include a human antibody, a humanized antibody; a chimeric antibody; a recombinant antibody; a single chain antibody; a diabody; a triabody; a tetrabody; a Fab fragment; a F(ab′)₂ fragment; an IgD antibody; an IgE antibody; an IgM antibody; an IgG1 antibody; an IgG2 antibody; an IgG3 antibody; or an IgG4 antibody, and fragments thereof. Also included are bispecific T cell engagers (BiTE®), bispecific T cell engagers having extensions, such as half-life extensions, for example HLE BiTEs, Heterolg BITE and others, chimeric antigen receptors (CARs, CAR Ts), and T cell receptors (TCRs).

As used herein, the term “antigen binding protein” is used in its broadest sense and means a protein comprising a portion that binds to an antigen or target and, optionally, a scaffold or framework portion that allows the antigen binding portion to adopt a conformation that promotes binding of the antigen binding protein to the antigen. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, e.g., Korndorfer et al., 2003, Proteins: Structure, Function, and Bioinformatics, 53(1):121-129; Roque et al., 2004, Biotechnol. Prog. 20:639-654. In addition, peptide antibody mimetics (“PAMs”) can be used, as well as scaffolds based on antibody mimetics utilizing fibronectin components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain can be done in accordance with the definitions of Kabat et al. in Sequences of Proteins of Immunological Interest, 5^{th} Ed., US Dept. of Health and Human Services, PHS, NIH, NIH Publication no. 91-3242, (1991). As desired, the CDRs can also be redefined according to an alternative nomenclature scheme, such as that of Chothia (see Chothia and Lesk, 1987, J. Mol. Biol. 196:901-917; Chothia et al., 1989, Nature 342:878-883 or Honegger and Pluckthun, 2001, J. Mol. Biol. 309:657-670).

In the context of the instant disclosure, an antigen binding protein is said to “specifically bind” or “selectively bind” its target antigen when the dissociation constant (K_{D}) is ≤10⁻⁸ M. The antibody specifically binds antigen with “high affinity” when the K_{D} is ≤5×10⁻⁹ M, and with “very high affinity” when the K_{D} is ≤5×10⁻¹⁰ M.

The term “antibody” includes reference to both glycosylated and non-glycosylated immunoglobulins of any isotype or subclass or to an antigen-binding region thereof that competes with the intact antibody for specific binding, unless otherwise specified. Additionally, the term “antibody” refers to an intact immunoglobulin or to an antigen binding portion thereof that competes with the intact antibody for specific binding, unless otherwise specified. Antigen binding portions can be produced by recombinant DNA techniques or by enzymatic or chemical cleavage of intact antibodies and can form an element of a protein of interest. Unless otherwise specified, antibodies include human, humanized, chimeric, multi-specific, monoclonal, polyclonal, heteroIgG, bispecific, and oligomers or antigen binding fragments thereof Antibodies include the lgG1-, lgG2- lgG3- or lgG4-type. Also included are proteins having an antigen binding fragment or region such as Fab, Fab′, F(ab′)2, Fv, diabodies, Fd, dAb, maxibodies, single chain antibody molecules, single domain V_{H}H, complementarity determining region (CDR) fragments, scFv, diabodies, triabodies, tetrabodies and polypeptides that contain at least a portion of an immunoglobulin that is sufficient to confer specific antigen binding to a target polypeptide.

An antigen binding protein can have one or more binding sites. If there is more than one binding site, the binding sites can be identical to one another or can be different. For example, a naturally occurring human immunoglobulin typically has two identical binding sites, while a “bispecific” or “bifunctional” antibody has two different binding sites.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, U.S. Patent Application Publication Nos. 2005/0202512, 2004/0202995, 2004/0038291, 2004/0009507, 2003/0039958, Ward et al., 1989, Nature 341:544-546).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83), U.S. Pat. Nos. 7,741,465, and 6,319,494 as well as Eshhar et al., 1997, Cancer Immunol Immunotherapy 45:131-136. An scFv retains the parent antibody's ability to specifically interact with target antigen.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48; and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

For purposes of clarity, and as described herein, it is noted that an antigen binding protein can, but need not, be of human origin (e.g., a human antibody), and in some cases will comprise a non-human protein, for example a rat or murine protein, and in other cases an antigen binding protein can comprise a hybrid of human and non-human proteins (e.g., a humanized antibody).

A protein of interest can comprise a human antibody. The term “human antibody” includes all antibodies that have one or more variable and constant regions derived from human immunoglobulin sequences. In one embodiment, all of the variable and constant domains are derived from human immunoglobulin sequences (a fully human antibody). Such antibodies can be prepared in a variety of ways, including through the immunization with an antigen of interest of a mouse that is genetically modified to express antibodies derived from human heavy and/or light chain-encoding genes, such as a mouse derived from a Xenomouse®, UltiMab™, or Velocimmune® system, or a rat derived from UniRat®. Phage-based approaches can also be employed.

Alternatively, a protein of interest can comprise a humanized antibody. A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. Examples of how to make humanized antibodies can be found in U.S. Pat. Nos. 6,054,297, 5,886,152 and 5,877,293.

Also included are modified proteins, such as are proteins modified chemically by a non-covalent bond, covalent bond, or both a covalent and non-covalent bond. Also included are proteins further comprising one or more post-translational modifications which may be made by cellular modification systems or modifications introduced ex vivo by enzymatic and/or chemical methods or introduced in other ways.

Proteins of interest may also include recombinant fusion proteins comprising, for example, a multimerization domain, such as a leucine zipper, a coiled coil, an Fc portion of an immunoglobulin, and the like. Also included are proteins comprising all or part of the amino acid sequences of differentiation antigens (referred to as CD proteins) or their ligands or proteins substantially similar to either of these.

In some embodiments, proteins of interest may include colony stimulating factors, such as granulocyte colony-stimulating factor (G-CSF). Such G-CSF agents include, but are not limited to, Neupogen® (filgrastim) and Neulasta® (pegfilgrastim). Also included are erythropoiesis stimulating agents (ESA), such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, epoetin omega, epoetin iota, tissue plasminogen activator, GLP-1 receptor agonists, as well as the molecules or variants or analogs thereof and biosimilars of any of the foregoing.

In some embodiments, proteins of interest may include proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments proteins of interest bind to one of more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In another embodiment, proteins of interest include abciximab, adalimumab, adecatumumab, aflibercept, alemtuzumab, alirocumab, anakinra, atacicept, basiliximab, belimumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, lxdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, and biosimilars of any of the foregoing.

Proteins of interest according to the invention encompass all of the foregoing and further include antibodies comprising 1, 2, 3, 4, 5, or 6 of the complementarity determining regions (CDRs) of any of the aforementioned antibodies. One or more CDRs can be incorporated into a molecule either covalently or noncovalently to make it an antigen binding protein. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest. Also included are variants that comprise a region that is 70% or more, especially 80% or more, more especially 90% or more, yet more especially 95% or more, particularly 97% or more, more particularly 98% or more, yet more particularly 99% or more identical in amino acid sequence to a reference amino acid sequence of a protein of interest. Identity in this regard can be determined using a variety of well-known and readily available amino acid sequence analysis software. Preferred software includes those that implement the Smith-Waterman algorithms, considered a satisfactory solution to the problem of searching and aligning sequences. Other algorithms also may be employed, particularly where speed is an important consideration. Commonly employed programs for alignment and homology matching of DNAs, RNAs, and polypeptides that can be used in this regard include FASTA, TFASTA, BLASTN, BLASTP, BLASTX, TBLASTN, PROSRCH, BLAZE, and MPSRCH, the latter being an implementation of the Smith-Waterman algorithm for execution on massively parallel processors made by MasPar.

Proteins of interest can also include genetically engineered receptors such as chimeric antigen receptors (CARs or CAR-Ts) and T cell receptors (TCRs), as well as other proteins comprising an antigen binding molecule that interacts with that targeted antigen. CARs can be engineered to bind to an antigen (such as a cell-surface antigen) by incorporating an antigen binding molecule that interacts with that targeted antigen. CARs typically incorporate an antigen binding domain (such as scFv) in tandem with one or more costimulatory (“signaling”) domains and one or more activating domains.

Preferably, the antigen binding molecule is an antibody fragment thereof, and more preferably one or more single chain antibody fragment (“scFv”). scFvs are preferred for use in chimeric antigen receptors because they can be engineered to be expressed as part of a single chain along with the other CAR components. See Krause et al., 1988, J. Exp. Med., 188(4): 619-626; Finney et al., 1998, J Immunol 161: 2791-2797.

Chimeric antigen receptors incorporate one or more costimulatory (signaling) domains to increase their potency. See U.S. Pat. Nos. 7,741,465, and 6,319,494, as well as Krause et al. and Finney et al. (supra), Song et al., 2012, Blood 119:696-706; Kalos et al., 2011, Sci Transl. Med. 3:95; Porter et al., 2011, N. Engl. J. Med. 365:725-33, and Gross et al., 2016, Annu. Rev. Pharmacol. Toxicol. 56:59-83. Suitable costimulatory domains can be derived from, among other sources, CD28, CD28T, OX40, 4-1BB/CD137, CD2, CD3 (alpha, beta, delta, epsilon, gamma, zeta), CD4, CDS, CD7, CD8, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD11a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, 41-BB, GADS, SLP-76, PAG/Cbp, CD19a, CD83 ligand, or fragments or combinations thereof. The costimulatory domain can comprise one or more of an extracellular portion, a transmembrane portion, and an intracellular portion.

CARs also include one or more activating domains. CD3 zeta is an element of the T cell receptor on native T cells and has been shown to be an important intracellular activating element in CARs.

CARs are transmembrane proteins, comprising an extracellular domain, typically containing an antigen binding protein that it is capable of recognizing and binding to the antigen of interest, and also including a “hinge” region. In addition is a transmembrane domain and an intracellular(cytoplasmic) domain.

The extracellular domain is beneficial for signaling and for an efficient response of lymphocytes to an antigen from any protein described herein or any combination thereof. The extracellular domain may be derived either from a synthetic or from a natural source, such as the proteins described herein. The extracellular domains often comprise a hinge portion. This is a portion of the extracellular domain, sometimes referred to as a “spacer” region. Hinges may be derived from the proteins as described herein, particularly the costimulatory proteins described above, as well as immunoglobulin (Ig) sequences or other suitable molecules to achieve the desired special distance from the target cell.

A transmembrane domain may be fused to the extracellular domain of the CAR. It can similarly be fused to the intracellular domain of the CAR. The transmembrane domain may be derived either from a synthetic or from a natural source, such as the proteins described herein, particularly the costimulatory proteins described above.

An intracellular (cytoplasmic) domain may be fused to the transmembrane domain and can provide activation of at least one of the normal effector functions of the immune cell. Effector function of a T cell, for example, may be cytolytic activity or helper activity including the secretion of cytokines. Intracellular domains can be derived from the proteins described herein, particularly from CD3.

An “Fc” region, as the term is used herein, comprises two heavy chain fragments comprising the C_{H}2 and C_{H}3 domains of an antibody. The two heavy chain fragments are held together by two or more disulfide bonds and by hydrophobic interactions of the C_{H}3 domains. Proteins of interest comprising an Fc region, including antigen binding proteins and Fc fusion proteins, form another aspect of the instant disclosure.

A “hemibody” is an immunologically functional immunoglobulin construct comprising a complete heavy chain, a complete light chain and a second heavy chain Fc region paired with the Fc region of the complete heavy chain A linker can, but need not, be employed to join the heavy chain Fc region and the second heavy chain Fc region. In particular embodiments, a hemibody is a monovalent form of an antigen binding protein disclosed herein. In other embodiments, pairs of charged residues can be employed to associate one Fc region with the second Fc region. A hemibody can be a protein of interest in the context of the instant disclosure.

A variety of known techniques can be utilized in making the polynucleotides, polypeptides, vectors, host cells, immune cells, compositions, and the like according to the invention.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

### Example 1

For routine culture, cells were cultivated in suspension, in selective medium. Cultures were maintained in either vented 125 mL or 250 mL Erlenmeyer shake flasks (Corning Life Sciences, Lowell, Mass.), 50 mL vented spin tubes (TPP, Trasadingen, Switzerland) or Axygen® 24-well Deep Well Plates (Axygen, Union City, Calif.) at 36° C., 5% CO₂ and 85% relative humidity. Erlenmeyer flasks were shaken at 120 rpm with a 25 mm orbital diameter in a large-capacity automatic CO₂ incubator (Thermo Fisher Scientific, Waltham, Mass.) and spin tubes were shaken at 225 rpm, 50 mm orbital diameter in a large capacity ISF4-X incubator (Kuhner AG, Basel, Switzerland).

### Adaption of CHO GSKO Host Cells to Medium Without IGF-1

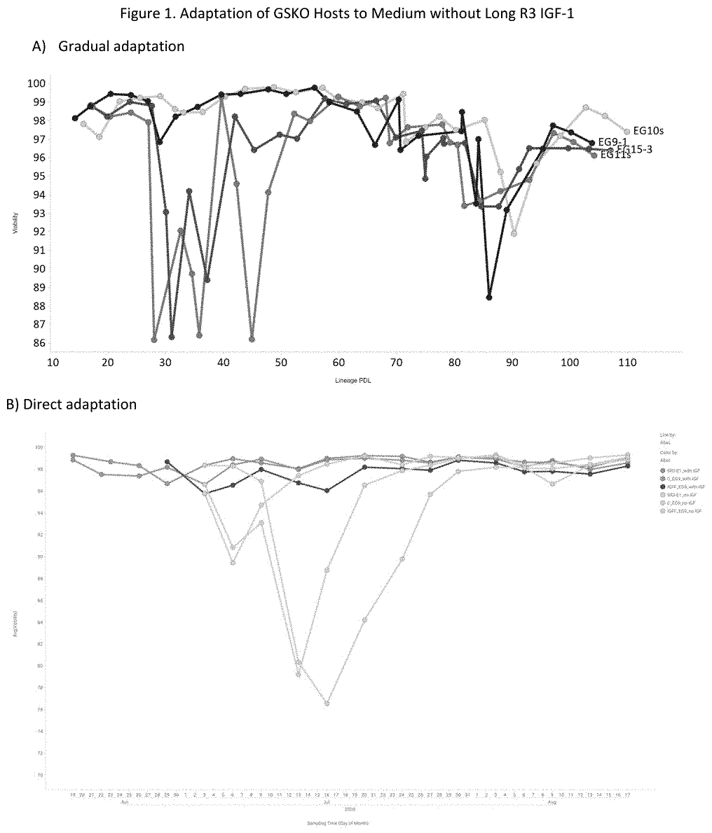

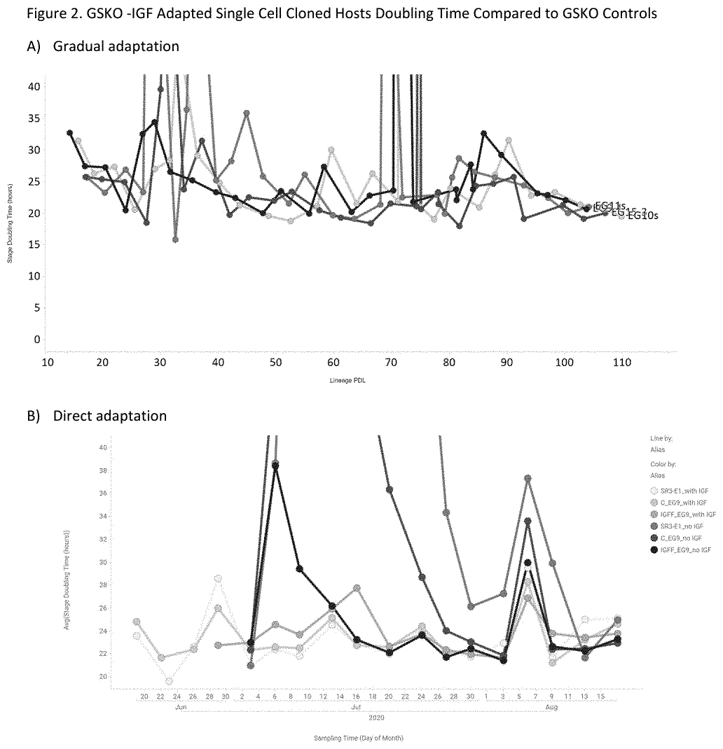

Glutamine Synthetase Knock-out (GSKO) host cell lines were adapted to media without IGF-1 (Long R3 IGF-1). These host cells were adapted using two different methods. The first method was gradual adaptation to a proprietary medium without IGF-1 (Long R3 IGF-1). This medium is not the standard non-selective host cell medium for GSKO host cells, thus the cells were first adapted to a different proprietary medium containing 100% IGF-1 (Long R3 IGF-1) medium and then IGF-1 was gradually withdrawn in the following increments: 75%, 50%, 25%, 10% and finally no IGF-1. The adaptation period extended over 110 population doubling levels (PDLs). See FIG. 1A. These adapted cell lines did not perform as well as the parental hosts with IGF-1 (data not shown), potentially due to the different osmolarity of the proprietary media. Thus, these cells were transitioned back to the platform non-selective medium for GSKO hosts without IGF-1. These hosts were all banked and single cell cloned using the Berkeley Lights (BLI) Beacon instrument. A total of 158 clones were obtained.

The second method was a direct adaptation. GSKO host cell lines were directly adapted to a proprietary media without IGF-1. These hosts were all banked and single cell cloned using the Berkeley Lights (BLI) Beacon instrument. Full recovery took ˜1.5-month time period. See FIG. 1B. A total of 44 clones were obtained.

### Single Cell Cloning Using the Berkeley Lights Procedure:

To ensure a clonally derived cell bank, the IGF⁻ adapted cell lines were single cell cloned using the Beacon instrument (Berkeley Lights, Emeryville, Calif.) under specific conditions. Proprietary media without Long R3 IGF-1 was used for the single cell cloning and scale up for both hosts. The Beacon instrument is a miniaturized cell culture platform that allows for single cell manipulation, cell culture, and productivity analysis. On this instrument, cells are cultured in isolation on a nanofluidic chip comprised of over 1000 individual vessels called “nanopens”, under controlled temperature, sterile environment, and continuous perfusion of growth medium Laminar flow is maintained throughout the culture duration to ensure no cross contamination. Opto-Electro Positioning (OEP) technology enables cell manipulation by using light-activated surface transistors to create a localized electric charge to repel cells. OEP is employed to gently guide individual cells in and out of the nanopens. Integrated microscopy capabilities allow for live cell imaging, loading of single cells, imaging, and exporting of cultures and is automated and controlled via software, ensuring traceability. Single cell clones were verified for single cell origin using repeated on-instrument microscopic imaging. See Le et al., 2020, Biotech J 15:1900247 and Le et al., 2018, Biotechnol Prog 34:1438-1446.

For IGF⁻ cell lines in the GSKO background, non-clonal cell pools were imported onto a new nanofluidic chip and single cells were isolated into individual nanopens using OEP. Integrated microscopic imaging was used to identify nanopens containing cells of single cell origin. Clones were cultured in individual nano pens for 3 days. Nanopens were then analyzed for growth. Cell populations were verified for clonal derivation and selected for varying growth profiles. Selected clones were independently exported off the chip into individual wells of a 96-well microtiter plate. Stringent quality control steps are built into this approach to ensure no detectable cross contamination. Statistical determination of clonal derivation demonstrates high assurance of isolation of a clonally derived cell line. See Le et al., 2020, Biotech J 15:1900247.

After export the single cell derived cell lines were scaled up with proprietary (+gln) non-selective growth medium without Long R3 IGF-1. The cell lines were passaged until they achieved >90% viability and stabilized growth. The cell lines were then banked into non-selective growth media without Long R3 IGF-1 and DMSO and frozen for long term storage at <−80° C.

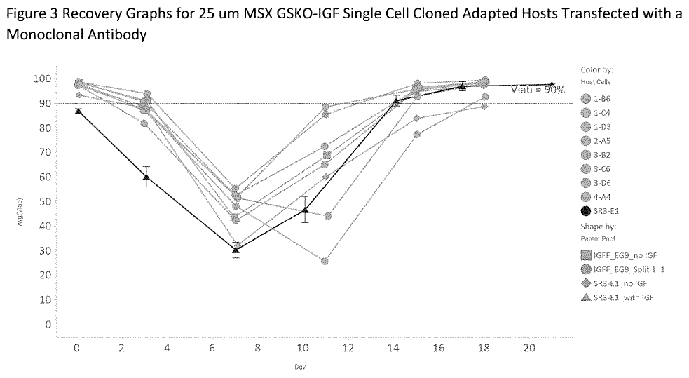

GSKO-IGF adapted single cell hosts had doubling times of 24 hours (FIG. 3A-B). The single cell hosts for further evaluation were initially narrowed based on performance in a stringent transfection/fed batch assessment and then further evaluated by transfection with a well-behaved monoclonal antibody as assessment in a 10-15 day fed batch assessment.

### Example 2

The ability of these IGF⁻ adapted host cells to grow and express therapeutics in the absence of IGF-1 supplementation was tested in transfection and 10D-15D FB (10 to 15 day fed batch) production experiments.

### Transfection and Recovery of Test Monoclonal Antibody Molecules in the Single Cell Cloned IGF− Adapted Cell Lines in CS9 GSKO Background

The GSKO IGF⁻ adapted hosts were tested by transfection and fed batch assessment in a proof of concept experiment with favorable results prior to single cell cloning (data not shown). For IGF⁻ single cell cloned adapted cell lines in the GSKO background, circular pGS1.1PB plasmid for a well-behaved monoclonal antibody in addition to a plasmid containing a proprietary ILT transposase were transfected using a platform long duration electroporation protocol. Transfected cell lines were recovered in proprietary non-selective media without Long R3 IGF-1 for 3 days at 36° C. and 5% CO₂. The transfected cells were passaged every 3 to 4 days in proprietary media +25 μM MSX selective growth media (-glutamine) without Long R3 IGF-1 at 36° C. and 5% CO₂ until they recovered to >90%. (FIG. 3). These GSKO IGF⁻ cell lines were then assessed in a 15D Fed batch production run.

### Fed Batch Production Cell Culture

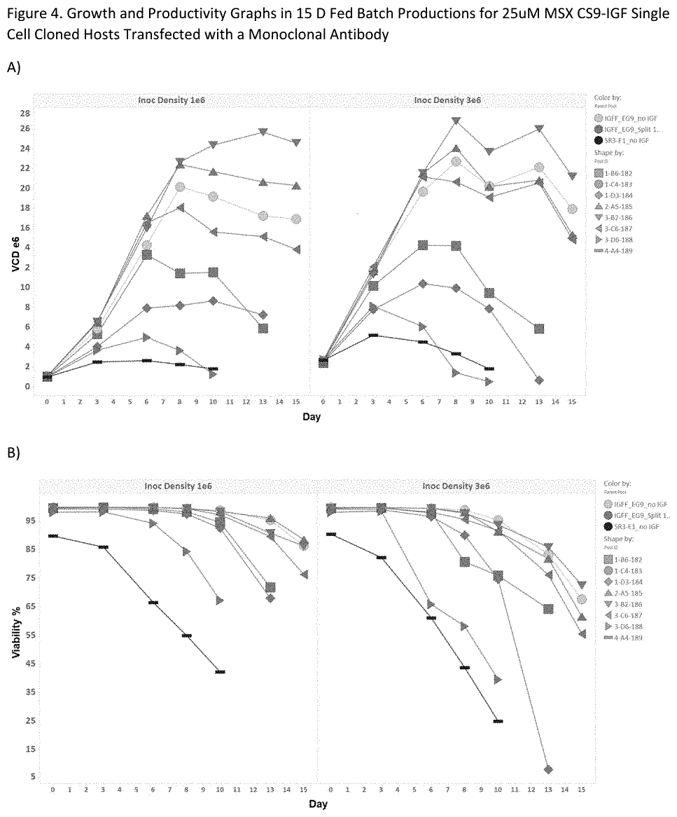

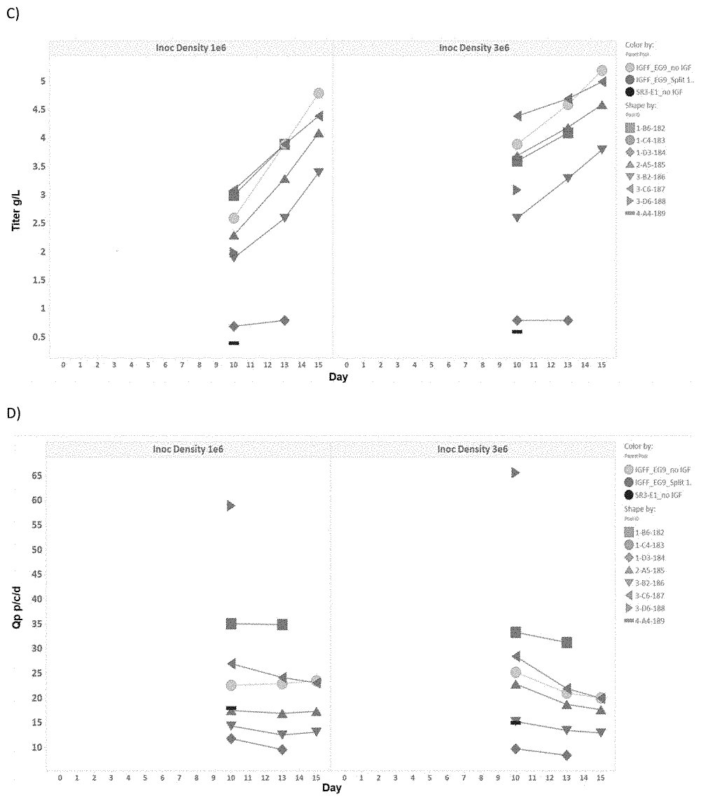

A 15 day fed batch production was done to assess growth and productivity of the transfected cell lines adapted to media without Long R3 IGF-1 in the GSKO background. The cultures were seeded at 3×10⁶ cells/mL (GSKO based) in a basal production medium without Long R3 IGF-1, and additional nutrients were fed on days 3, 6, 8, 10 and 13 for GSKO cultures. The GSKO cultures were harvested on day 15 or when viability dropped to 50-60% (FIGS. 4A-D). The production supernatants were analyzed for titer (Protein A HPLC).

The transfected cell lines demonstrated variable levels of growth and productivity with several in the range of GSKO cell lines with IGF-1.

### Example 3

The ability of these IGF⁻ adapted host cells to grow and express therapeutics of different modalities in the absence of IGF-1 supplementation was tested in transfection and 10D-15D FB (10 to 15 day fed batch) production experiments using the methods in Example 2 except different circular piggyBAC compatible ITR-containing vectors were used. The average values are from experiments run in duplicate. NA indicates that cultures were already harvested so no data is available.

BiTE—bispecific T-cell engager

Fusion—fusion protein

Hetero-Ig—hetero Ig bispecific antibody

mAb—monoclonal antibody

3-chain Ab—three chain asymmetrical antibody-like molecule

Tables 1-4 show IVCD, Viability %, Titer and Qp, respectively.

TABLE 1
IVCD

| | BiTE | | Fusion | | Hetero-IgG | | mAb | | 3-chain-Ab | |
|---|---|---|---|---|---|---|---|---|---|---|
| | −IGF | +IGF | −IGF | +IGF | −IGF | +IGF | −IGF | +IGF | −IGF | +IGF |
| Day 13 | 1039 | 1162 | 1081 | 1115 | 1311 | 1054 | 1086 | 1137 | 1165 | 1269 |
| Day 15 | 1221 | NA | 1210 | 1233 | 1540 | 1067 | NA | NA | 1365 | 1430 |

TABLE 2
Viability %

| | BiTE | | Fusion | | Hetero-IgG | | mAb | | 3-chain- Ab | |
|---|---|---|---|---|---|---|---|---|---|---|
| | −IGF | +IGF | −IGF | +IGF | −IGF | +IGF | −IGF | +IGF | −IGF | +IGF |
| Day 10 | 94 | 82 | 96 | 89 | 96 | 73 | 88 | 77 | 95 | 73 |
| Day 13 | 79 | 48 | 87 | 69 | 91 | 57 | 76 | 67 | 90 | 53 |
| Day 15 | 66 | NA | 0.7 | 55.4 | 84 | 49 | NA | NA | 79 | 44 |

TABLE 3
Titer

| | BiTE | | Fusion | | Hetero-IgG | | mAb | | 3-chain- Ab | |
|---|---|---|---|---|---|---|---|---|---|---|
| | −IGF | +IGF | −IGF | +IGF | −IGF | +IGF | −IGF | +IGF | −IGF | +IGF |
| Day 10 | 0.24 | 0.12 | 2.7 | 2.2 | 2.2 | 2.7 | 3.1 | 2.7 | 0.82 | 1.2 |
| Day 13 | 0.5 | 0.2 | 4.5 | 3.1 | 3.7 | 3.7 | 4.3 | 4.1 | 1.4 | 1.7 |
| Day 15 | 0.53 | NA | 2.5 | 4.8 | 4.4 | 2.6 | NA | NA | 1.6 | 1.5 |

TABLE 4
Qp

| | BiTE | | Fusion | | Hetero-IgG | | mAb | | 3-chain- Ab | |
|---|---|---|---|---|---|---|---|---|---|---|
| | −IGF | +IGF | −IGF | +IGF | −IGF | +IGF | −IGF | +IGF | −IGF | +IGF |
| Day 13 | 4.3 | 1.7 | 41.5 | 36 | 32 | 46 | 40 | 44.3 | 11.7 | 27 |
| Day 15 | 4.25 | NA | 20.6 | 39 | 30 | 65 | NA | NA | 11.5 | 25.3 |

The IGF− adapted cell lines demonstrated variable levels of growth and productivity but were comparable to GSKO cell lines with IGF-1.

### Example 4

An IGF⁻ adapted transfected host cell line was tested in a production scale 200 L bioreactor using a vector expressing a monoclonal antibody in a 15D FB (15 day fed batch) production experiment generally as described in Example 2.

Growth and titer were comparable to that seen in similar production runs with cell lines not adapted to IGF− conditions.

What is claimed is:

1. A method of producing a protein of interest from a mammalian cell culture comprising:
(a) culturing a mammalian cell expressing a protein of interest in a second cell culture media having 0.05 mg/L or less Insulin Like Growth Factor (IGF-1) to express the protein of interest, wherein the mammalian cell has been directly adapted to grow in a first cell culture media having 0.03 mg/L or less IGF-1 and comprises a heterologous nucleic acid encoding the protein of interest; and
(b) recovering the protein of interest produced by the mammalian cell.

2. The method of claim 1, wherein the second cell culture media contains less than 0.03 mg/L of IGF-1.

3. The method of claim 2, wherein the second cell culture media contains no IGF-1.

4. The method of claim 1, wherein the first cell culture media contains no IGF-1.

5. The method of claim 1, wherein the mammalian cell has a growth rate comparable to a mammalian cell of the same lineage that has not been directly adapted to media lacking IGF-1.

6. The method of claim 5, wherein the doubling time of the mammalian cell is less than 30 hours.

7. The method of claim 1, wherein the titer of the expressed protein of interest is at least 50 mg/L at day 10 of the culture.

8. The method of claim 1, wherein the protein of interest is an antigen binding protein.

9. The method of claim 8, wherein the protein of interest is selected from the group consisting of monoclonal antibodies, bi-specific T cell engagers, immunoglobulins, Fc fusion proteins and peptibodies.

10. The method of claim 1, wherein the mammalian cell culture process utilizes a fed-batch culture process, a perfusion culture process, or a combination thereof.

11. The method of claim 1, wherein the mammalian cell culture is established by inoculating a bioreactor of at least 100 L with at least 0.5×10⁶ to 3.0×10⁶ cells/mL in a serum-free culture media with 0.03 mg/L or less IGF-1.

12. The method of claim 1, wherein the mammalian cells are Chinese Hamster Ovary (CHO) cells.

13. The method of claim 12 wherein the CHO cells are deficient in dihydrofolate reductase (DHFR⁻) or a glutamine synthetase knock out (GSKO).

14. The method of claim 1, wherein the recovered protein of interest is purified and formulated in a pharmaceutically acceptable formulation.

15. The purified, formulated protein of interest of claim 14.

16. A method for directly adapting a mammalian cell to IGF⁻ media comprising:
a) culturing a population of mammalian cells in a cell culture medium comprising 0.03 mg/L or less IGF-1;
b) obtaining individual cells from the population of mammalian cells by single cell cloning;
c) expanding and passaging the individual cells until recovered to 90% or greater and a doubling time less than 30 hours.

17. The method of claim 16, wherein the cell culture medium comprises no IGF-1.

18. The method of claim 16, wherein the mammalian cells are Chinese Hamster Ovary (CHO) cells.

19. The method of claim 18, wherein the CHO cells are deficient in dihydrofolate reductase (DHFR⁻) or a glutamine synthetase knock out (GSKO).
